(19) United States
(12) Patent Application Publication
(10) Pub. No.: US 2011/0257237 A1
(43) Pub. Date: Oct. 20, 2011

(54) PROCESS FOR THE PREPARATION OF CRYSTALLINE FORMS OF SUNITINIB MALATE

(75) Inventors: Abhay Gaitonde, Maharashtra (IN); Vinayak Gore, Maharashtra (IN); Bharati Choudhari, Maharashtra (IN); Mahesh Hublikar, Maharashtra (IN); Prakash Bansode, Maharashtra (IN); Sunanda Phadtare, Maharashtra (IN)

(73) Assignee: GENERICS [UK] LIMITED, Potters Bar, Hertfordshire (GB)

(21) Appl. No.: 13/002,917

(22) PCT Filed: Jul. 9, 2009
(86) PCT No.: PCT/GB2009/050818

§ 371 Date: Jul. 11, 2011

(30) Foreign Application Priority Data
Jul. 10, 2008 (IN) 1193/KOL/2008

(51) Int. Cl. A61K 31/404; A61P 35/04; C07D 403/06

(52) U.S. Cl. 514/414; 548/465

(57) ABSTRACT

The present invention, relates to novel processes for the preparation of sunitinib malate formula (I), pharmaceutical compositions comprising said polymorph and the use of the said pharmaceutical compositions in the treatment of various forms of cancer.

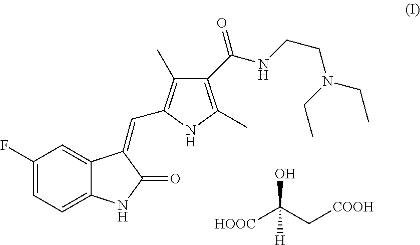

### FIELD OF THE INVENTION

The present invention relates to novel processes for the preparation of sunitinib malate form I, pharmaceutical compositions comprising said polymorph and the use of the said pharmaceutical compositions.

### BACKGROUND OF THE INVENTION

Sunitinib malate, represented by formula (I) and chemically named N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide 2(S)-hydroxybutanedioic acid, is a tyrosine kinase inhibitor (TKI) that targets and blocks the signaling pathways of multiple selected receptor tyrosine kinases (RTKs). Through competitive inhibition of ATP binding sites, sunitinib malate inhibits the TK activity of a group of closely related RTKs, all of which are involved in various human malignancies: the vascular endothelial growth factor receptors (VEGFR-1, -2, -3), the platelet derived growth factor receptors (PDGF-R), the stem cell factor (KIT), CSF-1R, Flt3, and RET. Sunitinib malate is therefore useful for the treatment of cancer and tumors. It is currently marketed for the treatment of unresectable and/or metastatic malignant gastrointestinal stromal tumor (GIST) and advanced and/or metastatic renal cell carcinoma (MRCC).

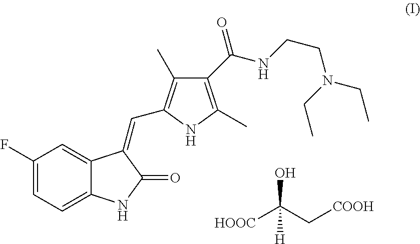

Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. The solubility of each polymorph may vary and consequently identifying the existence of polymorphs of an active pharmaceutical ingredient (API) is essential for providing pharmaceutical compositions with predictable solubility profiles. It is desirable to investigate all solid state forms of a drug, including all polymorphic forms. Polymorphic forms of a compound can be distinguished in a laboratory by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the API. For example, the solubility, stability, flowability, tractability and compressibility of the API as well as the safety and efficacy of the drug product can be dependent on the crystalline or polymorphic form.

Sunitinib malate was first described in U.S. Pat. No. 6,573,293. Processes for the synthesis of sunitinib are also described in the prior art. The prior art also describes the L-malate salt of sunitinib.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic.

Crystalline polymorphic forms I and II of sunitinib malate and methods of preparing the crystals are disclosed in prior art patent application WO 03/016305. In the prior art, L-malic acid is added to a solution of sunitinib free base in methanol and then the methanol is evaporated under reduced pressure resulting in a poorly crystalline orange solid. A further solvent, acetonitrile is added to the product to obtain a slurry which is heated and then cooled to obtain crystal form I. It will be apparent to the skilled person that this process is a multi-step process, thus making it more complicated.

In addition to the discovery of new polymorphic forms of a pharmaceutically useful compound, the development of improved methods for the preparation of known compounds and crystalline forms of said compounds is also very desirable. It is particularly desirable when the improvements in the process relate to improved purity or ease of manufacture of the desired compound or crystalline form.

### SUMMARY OF THE INVENTION

The inventors have developed methods for preparing anhydrous crystalline sunitinib malate form I, which overcome the disadvantages outlined above. In particular, the methods are simpler, generally employ fewer and less solvents, and generally result in sunitinib malate form I having greater than 99% chemical purity, without the need for additional steps that increase the cost and complexity of said methods.

Accordingly, there is provided in a first aspect a method for the preparation of sunitinib malate form I, comprising the steps of:

(i) mixing sunitinib with one or more solvents;

(ii) adding malic acid to the mixture from step (i); and

(iii) isolating a resultant solid from the mixture formed in step (ii).

In one embodiment, the sunitinib in step (i) is slurried in one or more solvents. Preferably the solvents are selected from polar solvents, such as polar protic or polar aprotic solvents and mixtures thereof.

Suitable polar protic solvents include for instance alcohols, water, carboxylic acids, amines and mixtures thereof. Preferred polar protic solvents are alcohols, preferably R¹OH, wherein R¹ is selected from an optionally substituted alkyl, aryl or arylalkyl group. Preferably the alcohol is monohydric. Preferably R¹ is an optionally substituted C₁₋₈ alkyl group, more preferably R¹ is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol, 1-octanol or a mixture thereof. Most preferably the alcohol is methanol.

Where the solvent is a polar protic solvent, preferably the sunitinib in step (i) is slurried in about 0.5 to 20 volumes of solvent, more preferably in about 5 to 15 volumes of solvent, most preferably in about 10 volumes of solvent.

Suitable polar aprotic solvents include for instance ethers such as tetrahydrofuran (THF), diethyl ether and methyl t-butyl ether; N,N-dimethylformamide (DMF); dimethylsulfoxide (DMSO); acetonitrile; esters such as ethyl acetate, isopropyl acetate, methyl propionate and methyl butyrate; ketones such as acetone, methyl ethyl ketone, methyl n-propyl ketone, diethyl ketone and cyclohexanone; and mixtures thereof. Preferred polar aprotic solvents are esters, ketones and mixtures thereof. Suitable esters include R^{a}COOR^{b}, wherein R^{a} and R^{b} are independently selected from optionally substituted alkyl, aryl or arylalkyl groups. Preferably R^{a} and R^{b} are independently optionally substituted C₁₋₈ alkyl groups, more preferably R^{a} and R^{b} are independently optionally substituted C₁₋₄ alkyl groups. Suitable ketones include R^{c}COR^{d}, wherein R^{c} and R^{d} are independently selected from optionally substituted alkyl, aryl or arylalkyl groups. Preferably R^{c} and R^{d} are independently optionally substituted C₁₋₈ alkyl groups, more preferably R^{c} and R^{d} are independently optionally substituted C₁₋₄ alkyl groups. Most preferred polar aprotic solvents are ethyl acetate and/or acetone.

Where the solvent is a polar aprotic solvent, preferably the sunitinib in step (i) is slurried in about 5 to 40 volumes of solvent, more preferably in about 10 to 30 volumes of solvent, most preferably in about 15 to 20 volumes of solvent.

In a preferred embodiment, the sunitinib in step (i) is slurried in one or more solvents selected from the group comprising acetone, methanol and ethyl acetate.

Preferably the sunitinib is slurried at about 0-100° C., more preferably the sunitinib is slurried at about 5-50° C., most preferably the sunitinib is slurried at about 15-35° C. In such embodiments, one or more of the steps can be performed individually at about 15-35° C.

Alternatively, the sunitinib in step (i) is dissolved in one or more solvents. Preferably the solvents are selected from polar solvents, such as polar protic or polar aprotic solvents and mixtures thereof.

Suitable polar protic solvents include for instance alcohols, water, carboxylic acids, amines and mixtures thereof. Preferred polar protic solvents are alcohols, preferably R²OH, wherein R² is selected from an optionally substituted alkyl, aryl or arylalkyl group. Preferably the alcohol is monohydric. Preferably R² is an optionally substituted C₁₋₈ alkyl group, more preferably R² is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol, 1-octanol or a mixture thereof. Most preferably the alcohol is methanol.

Where the solvent is a polar protic solvent, preferably the sunitinib in step (i) is dissolved in about 5 to 100 volumes of solvent, more preferably in about 10 to 50 volumes of solvent, most preferably in about 20 volumes of solvent.

Suitable polar aprotic solvents include for instance ethers such as tetrahydrofuran (THF), diethyl ether and methyl t-butyl ether; N,N-dimethylformamide (DMF); dimethylsulfoxide (DMSO); acetonitrile; esters such as ethyl acetate, isopropyl acetate, methyl propionate and methyl butyrate; ketones such as acetone, methyl ethyl ketone, methyl n-propyl ketone, diethyl ketone and cyclohexanone; and mixtures thereof. Preferred polar aprotic solvents are esters, ketones and mixtures thereof. Suitable esters include R^{e}COOR^{f}, wherein R^{e} and R^{f} are independently selected from optionally substituted alkyl, aryl or arylalkyl groups. Preferably R^{e} and R^{f} are independently optionally substituted C₁₋₈ alkyl groups, more preferably R^{e} and R^{f} are independently optionally substituted C₁₋₄ alkyl groups. Suitable ketones include R^{g}COR^{h}, wherein R^{g} and R^{h} are independently selected from optionally substituted alkyl, aryl or arylalkyl groups. Preferably R^{g} and R^{h} are independently optionally substituted C₁₋₈ alkyl groups, more preferably R^{g} and R^{h} are independently optionally substituted C₁₋₄ alkyl groups. Most preferred are ethyl acetate and/or acetone.

Where the solvent is a polar aprotic solvent, preferably the sunitinib in step (i) is dissolved in about 10 to 200 volumes of solvent, more preferably in about 20 to 100 volumes of solvent. Where the solvent is an ester, most preferably the sunitinib in step (i) is dissolved in about 60 volumes of solvent. Where the solvent is a ketone, most preferably the sunitinib in step (i) is dissolved in about 30 volumes of solvent.

In a preferred embodiment, the sunitinib in step (i) is dissolved in one or more solvents selected from the group comprising acetone, methanol and ethyl acetate.

Preferably the sunitinib is dissolved at about 0-200° C., more preferably the sunitinib is dissolved at about 20-150° C., more preferably still the sunitinib is dissolved at about 30-100° C., and most preferably the sunitinib is dissolved at about 50-80° C. In a particularly preferred embodiment, the sunitinib is dissolved at about reflux temperature.

A further preferred embodiment of the first aspect of the present invention provides that the malic acid added in step (ii) is L- or D-malic acid, most preferably L-malic acid.

In yet another embodiment, the malic acid is dissolved in one or more solvents, preferably selected from polar protic or polar aprotic solvents and mixtures thereof, before adding to the mixture formed in step (i). Preferably the one or more solvents for dissolving the malic acid are selected from polar prone solvents. Suitable polar protic solvents for dissolving the malic acid include for instance water and alcohols, preferably R³OH, wherein R³ is selected from an optionally substituted alkyl, aryl or arylalkyl group. Preferably the alcohol is monohydric. Preferably R³ is an optionally substituted C₁₋₈ alkyl group, more preferably R³ is an optionally substituted C₁₋₄ alkyl group. Preferably the alcohol is methanol, ethanol, 1-propanol, isopropanol, 1-butanol, 2-methyl-1-propanol, t-butanol, 1-pentanol, cyclopentanol, 1-hexanol, cyclohexanol, 1-heptanol, 1-octanol or a mixture thereof. More preferably the polar prone solvent is water, methanol or a mixture thereof. Most preferably the polar prone solvent is methanol.

Alternatively, the one or more solvents for dissolving the malic acid may be selected from polar aprotic solvents. Suitable polar aprotic solvents include for instance ethers such as tetrahydrofuran (THF), diethyl ether and methyl t-butyl ether; N,N-dimethylformamide (DMF); dimethylsulfoxide (DMSO); acetonitrile; esters such as ethyl acetate, isopropyl acetate, methyl propionate and methyl butyrate; ketones such as acetone, methyl ethyl ketone, methyl n-propyl ketone, diethyl ketone and cyclohexanone; and mixtures thereof. Preferred polar aprotic solvents are esters, ketones and mixtures thereof. Suitable esters include RⁱCOOR^{j}, wherein Rⁱ and R^{j} are independently selected from optionally substituted alkyl, aryl or arylalkyl groups. Preferably Rⁱ and R^{j} are independently optionally substituted C₁₋₈ alkyl groups, more preferably Rⁱ and R^{j} are independently optionally substituted C₁₋₄ alkyl groups. Suitable ketones include R^{k}COR^{l}, wherein R^{k} and R^{l} are independently selected from optionally substituted alkyl, aryl or arylalkyl groups. Preferably R^{k} and R^{l} are independently optionally substituted C₁₋₈ alkyl groups, more preferably R^{k} and R^{l} are independently optionally substituted C₁₋₄ alkyl groups. Most preferred is acetone.

Preferably the malic acid is dissolved in about 0.1 to 100 volumes of solvent. More preferably the malic acid is dissolved in about 1 to 10 volumes of solvent. Most preferably the malic acid is dissolved in about 4 volumes of solvent.

In a further embodiment, the malic acid is added to the sunitinib in step (ii) whilst the mixture from step (i) is agitated or sonicated, preferably agitated by stirring.

In yet another embodiment of the first aspect of the present invention, the malic acid is added to the sunitinib in step (ii) at a rate of less than 10 equivalents of malic acid per minute. Preferably the malic acid is added at a rate of less than 1 equivalent per minute. More preferably the malic acid is added at a rate of less than 0.1 equivalents per minute. Most preferably the malic acid is added at a rate of about 0.05 equivalents per minute.

In a preferred embodiment of the first aspect of the present invention, the resultant solid isolated in step (iii) is sunitinib malate form I. Preferably the sunitinib malate form I isolated in step (iii) has a chemical and/or polymorphic purity of greater than 95%, preferably greater than 99%, more preferably greater than 99.5%, most preferably greater than 99.7%.

A second aspect of the present invention relates to a method for the preparation of sunitinib malate form I, comprising the steps of:

(i) mixing malic acid with one or more solvents;

(ii) adding sunitinib to the mixture from step (i); and

(iii) isolating a resultant solid from the mixture formed in step (ii).

In one embodiment of the second aspect of the present invention, the sunitinib is dissolved or slurried in one or more solvents before addition to the mixture from step (i). The one or more solvents may be independently selected from any of those listed as being suitable or preferred for slurrying or dissolving the sunitinib in step (i) of the first aspect of the invention.

For instance, the sunitinib in step (ii) of the second aspect of the invention may be slurried in one or more solvents selected from the group comprising acetone, methanol and ethyl acetate.

Where the sunitinib is slurried in the second aspect of the invention, preferably the sunitinib is slurried at about 0-100° C., more preferably the sunitinib is slurried at about 5-50° C., most preferably the sunitinib is slurried at about 15-35° C.

Where the solvent is a polar protic solvent, preferably the sunitinib in step (ii) of the second aspect of the invention is slurried in about 0.5 to 20 volumes of solvent, more preferably in about 5 to 15 volumes of solvent, most preferably in about 10 volumes of solvent.

Alternatively, where the solvent is a polar aprotic solvent, preferably the sunitinib in step (ii) of the second aspect of the invention is slurried in about 5 to 40 volumes of solvent, more preferably in about 10 to 30 volumes of solvent, most preferably in about 15 to 20 volumes of solvent.

Alternatively, the sunitinib in step (ii) of the second aspect of the invention may be dissolved in one or more solvents selected from the group comprising acetone, methanol and ethyl acetate.

Where the sunitinib is dissolved in the second aspect of the invention, preferably the sunitinib is dissolved at about 0-200° C., more preferably the sunitinib is dissolved at about 20-150° C., more preferably still the sunitinib is dissolved at about 30-100° C., and most preferably the sunitinib is dissolved at about 50-80° C. In a particularly preferred embodiment, the sunitinib is dissolved at about reflux temperature.

Where the solvent is a polar protic solvent, preferably the sunitinib in step (ii) of the second aspect of the invention is dissolved in about 5 to 100 volumes of solvent, more preferably in about 10 to 50 volumes of solvent, most preferably in about 20 volumes of solvent.

Where the solvent is a polar aprotic solvent, preferably the sunitinib in step (ii) of the second aspect of the invention is dissolved in about 10 to 200 volumes of solvent, more preferably in about 20 to 100 volumes of solvent. Where the solvent is an ester, most preferably the sunitinib in step (ii) is dissolved in about 60 volumes of solvent. Where the solvent is a ketone, most preferably the sunitinib in step (ii) is dissolved in about 30 volumes of solvent.

In another embodiment of the second aspect of the present invention, the malic acid in step (i) is L- or D-malic acid. Preferably the malic acid is L-malic acid.

In yet another embodiment, the malic acid is dissolved in the one or more solvents in step (i). The one or more solvents may be independently selected from any of those listed as being suitable or preferred for dissolving the malic acid in step (ii) of the first aspect of the invention. Preferably the malic acid is dissolved in methanol.

Where the malic acid is dissolved in the second aspect of the invention, in one embodiment the malic acid is dissolved at about 0-100° C., more preferably the malic acid is dissolved at about 5-50° C., most preferably the malic acid is dissolved at about 15-35° C. In an alternate embodiment, the malic acid is dissolved at about 0-200° C., more preferably the malic acid is dissolved at about 20-150° C., more preferably still the malic acid is dissolved at about 30-100° C., and most preferably the malic acid is dissolved at about 50-80° C. In a particularly preferred embodiment, the malic acid is dissolved at about reflux temperature.

Preferably, where the malic acid is dissolved in the second aspect of the invention, the malic acid is dissolved in about 0.1 to 100 volumes of solvent. More preferably the malic acid is dissolved in about 1 to 10 volumes of solvent. Most preferably the malic acid is dissolved in about 4 volumes of solvent.

In a further embodiment of the second aspect of the present invention, the sunitinib is added to the malic acid in step (ii) whilst the mixture from step (i) is agitated or sonicated, preferably agitated by stirring.

In yet another embodiment of the second aspect of the present invention, the sunitinib is added to the malic acid in step (ii) at a rate of less than 10 equivalents of sunitinib per minute. Preferably the sunitinib is added at a rate of less than 1 equivalent per minute. More preferably the sunitinib is added at a rate of less than 0.1 equivalents per minute. Most preferably the sunitinib is added at a rate of about 0.05 equivalents per minute.

In a preferred embodiment of the second aspect of the present invention, the resultant solid isolated in step (iii) is sunitinib malate form I. Preferably the sunitinib malate form I isolated in step (iii) has a chemical and/or polymorphic purity of greater than 95%, preferably greater than 99%, more preferably greater than 99.5%, most preferably greater than 99.7%.

In one embodiment according to either the first or second aspects of the present invention, after the addition of the malic acid or the sunitinib in step (ii), a further step (ii-a) of heating the mixture is performed before the isolation of step (iii) occurs. Preferably the mixture is heated to about 40-200° C., more preferably the mixture is heated to about 45-120° C., and more preferably still the mixture is heated to about 50-80° C. In a particularly preferred embodiment, in step (ii-a) the mixture is heated to about the reflux temperature of the solvent or solvent mixture.

Preferably in step (ii-a) the mixture is heated for about 5-120 minutes, more preferably the mixture is heated for about 10-60 minutes, more preferably still the mixture is heated for about 15-30 minutes.

In another embodiment according to either the first or second aspects of the present invention, after the addition of the malic acid or the sunitinib in step (ii) and, if present, after the heating of the mixture in step (ii-a), a further step (ii-b) of allowing the mixture to stand for a period of at least 5 minutes is performed before the isolation of step (iii) occurs. Preferably the mixture is allowed to stand for a period of about 5-120 minutes, more preferably the mixture is allowed to stand for a period of about 10-60 minutes, more preferably still the mixture is allowed to stand for a period of about 15-30 minutes.

Preferably during step (ii-b) the mixture is kept at a temperature of about 0-40° C. More preferably during step (ii-b) the mixture is kept at a temperature of about 20-35° C.

Optionally during step (ii-b) the mixture is agitated or sonicated, preferably agitated by stirring.

In an alternate embodiment according to either the first or second aspects of the present invention, after the addition of the malic acid or the sunitinib in step (ii), a further step of stirring the mixture is performed until a slurry is formed. In preferred embodiments, the resultant slurry is heated or refluxed. Preferably the resultant slurry is heated to about 40-200° C., more preferably the resultant slurry is heated to about 45-120° C., and more preferably still the resultant slurry is heated to about 50-80° C. In a particularly preferred embodiment, the resultant slurry is heated to about the reflux temperature of the solvent or solvent mixture.

Preferably the resultant slurry is heated for about 5-120 minutes, more preferably the resultant slurry is heated for about 10-60 minutes, more preferably still the resultant slurry is heated for about 15-30 minutes.

Preferably the slurry is allowed to cool to about 0-40° C. or 0-35° C. More preferably the slurry is allowed to cool to about 15-35° C.

Preferably the slurry is stirred for a defined period of time. Preferably the defined period of time is about 5-120 minutes, more preferably the defined period of time is about 10-60 minutes, more preferably still the defined period of time is about 15-30 minutes.

In a particularly preferred embodiment according to either the first or second aspects of the present invention, the solid sunitinib malate form I from step (iii) is isolated by means of filtration and in some embodiments may be washed, preferably with the same solvent(s) as used in step (i). The sunitinib malate form I may further be dried until a constant weight is achieved under conditions that do not degrade the isolated solid sunitinib malate form I. Preferably the drying occurs at about 40° C., preferably under reduced pressure.

In a third aspect according to the invention there is provided a method for the preparation of sunitinib malate form I, comprising the steps of:
(i) dissolving sunitinib in a solvent system wherein said solvent system comprises one or more solvents chosen from the group comprising acetone, methanol and ethyl acetate;
(ii) adding malic acid to the solution from step (i);
(iii) stirring the resultant solution for a defined period of time; and
(iv) isolating the resultant solid sunitinib malate form I from the mixture formed in step (iii).

Preferably the sunitinib is dissolved in the solvent system from step (i) at a temperature of about 0-200° C., more preferably the sunitinib is dissolved at about 20-150° C., more preferably still the sunitinib is dissolved at about 30-100° C., and most preferably the sunitinib is dissolved at about 50-80° C. In a particularly preferred embodiment, the sunitinib is dissolved in the solvent system from step (i) under reflux conditions.

Where the solvent system is methanol, preferably the sunitinib in step (i) of the third aspect of the invention is dissolved in about 5 to 100 volumes of solvent, more preferably in about 10 to 50 volumes of solvent, most preferably in about 20 volumes of solvent.

Where the solvent system is acetone and/or ethyl acetate, preferably the sunitinib in step (i) of the third aspect of the invention is dissolved in about 10 to 200 volumes of solvent, more preferably in about 20 to 100 volumes of solvent. Where the solvent system is ethyl acetate, most preferably the sunitinib in step (i) is dissolved in about 60 volumes of solvent. Where the solvent system is acetone, most preferably the sunitinib in step (i) is dissolved in about 30 volumes of solvent.

A further preferred embodiment of the third aspect of the present invention provides that the malic acid in step (ii) is L- or D-malic acid, most preferably L-malic acid.

In a particularly preferred embodiment of the third aspect of the present invention, the malic acid is added slowly in step (ii). Preferably the malic acid is added to the sunitinib in step (ii) at a rate of less than 10 equivalents of malic acid per minute. More preferably the malic acid is added at a rate of less than 1 equivalent per minute. More preferably still the malic acid is added at a rate of less than 0.1 equivalents per minute. Most preferably the malic acid is added at a rate of about 0.05 equivalents per minute.

In yet another embodiment of the third aspect of the present invention, the malic acid is dissolved in one or more organic solvents, before adding to the mixture formed in step (i). The one or more organic solvents may be independently selected from any of those listed as being suitable or preferred for dissolving the malic acid in step (ii) of the first aspect of the present invention. Preferably the malic acid is dissolved in methanol.

Preferably, where the malic acid is dissolved in the third aspect of the invention, the malic acid is dissolved in about 0.1 to 100 volumes of solvent. More preferably the malic acid is dissolved in about 1 to 10 volumes of solvent. Most preferably the malic acid is dissolved in about 4 volumes of solvent.

In a further embodiment of the third aspect of the present invention, the malic acid is added to the sunitinib in step (ii) whilst the mixture from step (i) is agitated or sonicated, preferably agitated by stirring.

In further preferred embodiments of the third aspect of the present invention, there are provided processes according to the invention wherein the solution from step (iii) may be stirred at elevated temperatures, such as about 40-200° C., more preferably about 45-120° C., and more preferably still about 50-80° C. Most preferably the solution from step (iii) is stirred at about reflux temperatures. Preferably the solution from step (iii) is stirred at elevated temperatures for about 5-120 minutes, more preferably for about 10-60 minutes, more preferably still for about 15-30 minutes.

In a particularly preferred embodiment of the third aspect of the present invention, the mixture in step (iii) is allowed to cool to about 0-40° C. or 0-35° C. More preferably the slurry is allowed to cool to about 15-35° C. Most preferably the mixture in step (iii) is allowed to cool to ambient temperature.

In one embodiment of the third aspect of the present invention, the defined period of time in step (iii) is about 5-240 minutes, more preferably the defined period of time is about 20-120 minutes, more preferably still the defined period of time is about 30-60 minutes.

In a particularly preferred embodiment of the third aspect of the present invention, solid sunitinib malate form I from step (iv) is isolated by means of filtration and in some embodiments may be washed, preferably with the same solvent(s) as used in step (i). The sunitinib malate form I may further be dried until a constant weight is achieved under conditions that do not degrade the isolated solid sunitinib malate form I. Preferably the drying occurs at about 15-35° C., most preferably at about 40° C., preferably under reduced pressure, most preferably in a vacuum or partial vacuum.

In one embodiment the sunitinib malate form I isolated in step (iv) of the third aspect of the present invention has a chemical and/or polymorphic purity of greater than 95%, preferably greater than 99%, more preferably greater than 99.5%, most preferably greater than 99.7%.

In a fourth aspect according to the invention there is provided an alternative method for the preparation of sunitinib malate form I, comprising the steps of:
(i) slurrying sunitinib in a solvent system wherein said solvent system comprises one or more solvents chosen from the group comprising acetone, methanol and ethyl acetate;
(ii) adding malic acid to the slurry from step (i);
(iii) stirring the slurry for a defined period of time; and
(iv) isolating the resultant solid sunitinib malate form I.

A particularly preferred embodiment of the fourth aspect of the present invention provides a method wherein one or more of the individual steps (including all of the individual steps) can be performed individually at about 0-100° C., more preferably at about 5-50° C., and most preferably at about 15-35° C.

Where the solvent system is methanol, preferably the sunitinib in step (i) of the fourth aspect of the invention is slurried in about 0.5 to 20 volumes of solvent, more preferably in about 5 to 15 volumes of solvent, most preferably in about 10 volumes of solvent.

Alternatively, where the solvent system is acetone and/or ethyl acetate, preferably the sunitinib in step (i) of the fourth aspect of the invention is slurried in about 5 to 40 volumes of solvent, more preferably in about 10 to 30 volumes of solvent, most preferably in about 15 to 20 volumes of solvent.

A further preferred embodiment provides that the malic acid in step (ii) is L- or D-malic acid, most preferably L-malic acid.

In a particularly preferred embodiment, the malic acid is added in step (ii) at ambient temperatures, preferably at about 15-35° C.

In another embodiment of the fourth aspect of the present invention, the malic acid is added to the sunitinib in step (ii) at a rate of less than 10 equivalents of malic acid per minute. Preferably the malic acid is added at a rate of less than 1 equivalent per minute. More preferably the malic acid is added at a rate of less than 0.1 equivalents per minute. Most preferably the malic acid is added at a rate of about 0.05 equivalents per minute.

In yet another embodiment of the fourth aspect of the present invention, the malic acid is dissolved in one or more solvents, before adding to the slurry formed in step (i). The one or more solvents may be independently selected from any of those listed as being suitable or preferred for dissolving the malic acid in step (ii) of the first aspect of the present invention. Preferably the malic acid is dissolved in methanol.

Preferably, where the malic acid is dissolved in the fourth aspect of the invention, the malic acid is dissolved in about 0.1 to 100 volumes of solvent. More preferably the malic acid is dissolved in about 1 to 10 volumes of solvent. Most preferably the malic acid is dissolved in about 4 volumes of solvent.

Preferably the slurry in step (iii) of the fourth aspect of the present invention is stirred at about 0-40° C. or 0-35° C. More preferably the slurry is stirred at about 15-35° C.

Preferably the defined period of time in step (iii) of the fourth aspect of the present invention is about 5-120 minutes, more preferably the defined period of time is about 10-60 minutes, more preferably still the defined period of time is about 15-30 minutes.

In a particularly preferred embodiment of the fourth aspect of the present invention, the resultant solid sunitinib malate form I is isolated by means of filtration and in some embodiments may be washed, preferably with the same solvent(s) as used in step (i). The sunitinib malate form I may further be dried until a constant weight is achieved under conditions that do not degrade the isolated solid sunitinib malate form I. Preferably the drying occurs at about 15-35° C., most preferably at about 40° C., preferably under reduced pressure, most preferably under vacuum or partial vacuum.

In one embodiment the sunitinib malate form I isolated in step (iv) of the fourth aspect of the present invention has a chemical and/or polymorphic purity of greater than 95%, preferably greater than 99%, more preferably greater than 99.5%, most preferably greater than 99.7%.

A fifth aspect of the present invention provides sunitinib malate form I when prepared according to any of the aspects or embodiments according to the invention. Preferably the sunitinib malate form I has a chemical and/or polymorphic purity of greater than 95%, preferably greater than 99%, more preferably greater than 99.5%, most preferably greater than 99.7%. Preferably the chemical purity is as measured by HPLC. Preferably the polymorphic purity is as measured by XRPD or DSC, preferably XRPD.

In a preferred embodiment of the fifth aspect of the present invention, the sunitinib malate form I is for use in medicine.

A sixth aspect according to the invention provides a pharmaceutical composition comprising sunitinib malate form I prepared according to any of the aspects and embodiments according to the invention and disclosed herein, such as sunitinib malate form I according to the fifth aspect of the present invention. Preferably the pharmaceutical composition further comprises one or more pharmaceutically acceptable excipients or diluents.

Preferably the sunitinib malate form I according to the fifth aspect of the present invention or the pharmaceutical composition according to the sixth aspect of the present invention is provided for use in the treatment of cancer, in particular in the treatment of cancer and tumors, and most preferably for the treatment of unresectable and/or metastatic malignant gastrointestinal stromal tumor (GIST) or advanced and/or metastatic renal cell carcinoma (MRCC).

Alternately or in addition, the sunitinib malate form I according to the fifth aspect of the present invention or the pharmaceutical composition according to the sixth aspect of the present invention may be provided for use in the treatment of disorders related to abnormal protein kinase (PK) activity.

A seventh aspect according to the invention provides the use of sunitinib malate form I according to the fifth aspect of the present invention for the manufacture of a medicament. In one embodiment, said medicament is for the treatment of a tumor. Preferably said medicament is for the treatment of cancer. More preferably said medicament is for the treatment of unresectable and/or metastatic malignant gastrointestinal stromal tumor (GIST) or advanced and/or metastatic renal cell carcinoma (MRCC).

An eighth aspect according to the invention provides a method of treatment, comprising administering to a patient in need thereof a therapeutically effective amount of sunitinib malate form I according to the fifth aspect of the present invention. In one embodiment, said method is for the treatment of a tumor. Preferably said method is for the treatment of cancer. More preferably said method is for the treatment of unresectable and/or metastatic malignant gastrointestinal stromal tumor (GIST) or advanced and/or metastatic renal cell carcinoma (MRCC). In another embodiment of the eighth aspect of the present invention, the patient is a mammal. Preferably the patient is a human.

In an alternate or additional embodiment according to either the seventh or the eighth aspects of the present invention, the medicament or method may be for the treatment of disorders related to abnormal protein kinase (PK) activity.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### BRIEF DESCRIPTION OF THE ACCOMPANYING FIGURES

FIG. 1 describes the X-ray powder diffraction (XRPD) of sunitinib malate form I as prepared according to the invention.

FIG. 2 describes the prior art X-ray powder diffraction (XRPD) of sunitinib malate form I as disclosed in WO 03/016305, which is herein incorporated in its entirety by reference.

### DETAILED DESCRIPTION OF THE INVENTION

As used herein, the term ‘sunitinib’ preferably relates to the free base form, but may also relate to any other form including different salts, crystalline forms, amorphous form etc. unless otherwise stated.

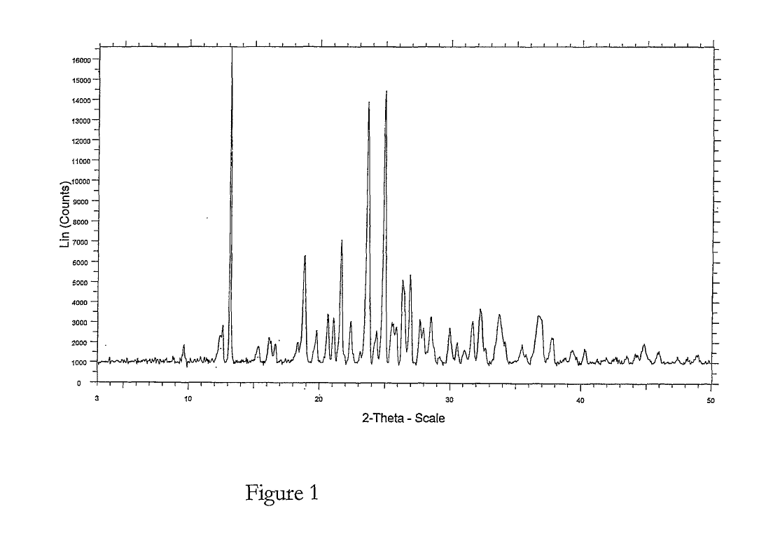

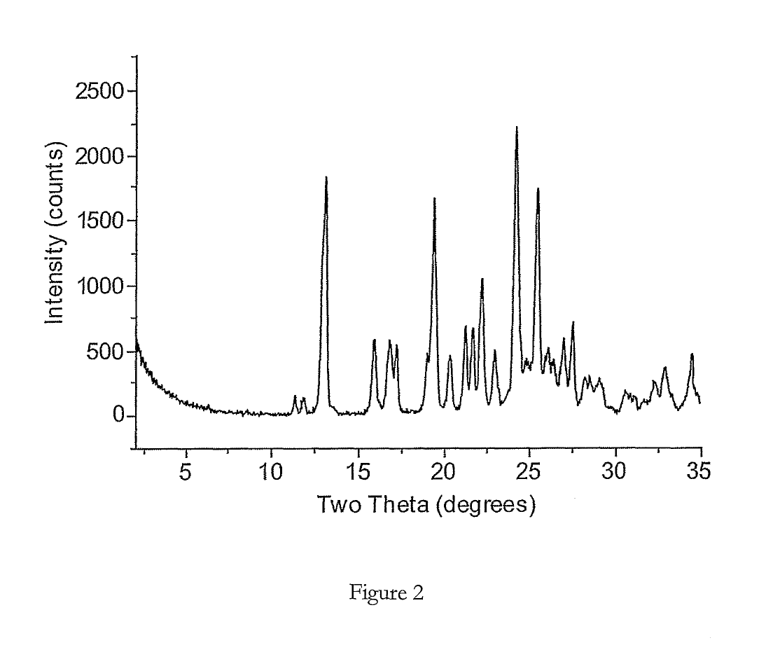

As used herein, crystalline form I of sunitinib malate is as defined in WO 03/016305, i.e. characterized by an X-ray diffraction pattern having peaks at 20 values at about 13.2, 19.4, 24.2 and 25.5° 2θ. Preferably crystalline form I of sunitinib malate has an X-ray diffraction pattern substantially as illustrated in FIG. 1 and/or FIG. 2.

As used herein, the term ‘mixture’ may relate to any combination of substances, for example, a solution, a partial solution where the solute is not fully dissolved, a solution of two or more miscible liquids, a slurry and a suspension of any type may all be included,

For the purposes of the present invention, one ‘volume’ or ‘vol’ in relation to a liquid (such as a solvent) refers to 1 ml of said solvent for each gram of sunitinib used in the process.

As used herein, the term ‘ambient temperature’ refers to a temperature range from about 15° C. to about 35° C., preferably from about 22° C. to about 27° C.

As used herein, an ‘alkyl’ group is defined as a monovalent saturated hydrocarbon, which may be straight-chained or branched, or be or include cyclic groups. An alkyl group may optionally include one or more heteroatoms N, O or S in its carbon skeleton. Examples of alkyl groups are methyl, ethyl, n-propyl, i-propyl, n-butyl, i-butyl, t-butyl and n-pentyl groups. Preferably an alkyl group is straight-chained or branched and does not include any heteroatoms in its carbon skeleton. Preferably an alkyl group is a C₁-C₁₂ alkyl group, which is defined as an alkyl group containing from 1 to 12 carbon atoms. More preferably an alkyl group is a C₁-C₆ alkyl group, which is defined as an alkyl group containing from 1 to 6 carbon atoms. An ‘alkylene’ group is similarly defined as a divalent alkyl group.

An ‘alkenyl’ group is defined as a monovalent hydrocarbon, which comprises at least one carbon-carbon double bond, which may be straight-chained or branched, or be or include cyclic groups. An alkenyl group may optionally include one or more heteroatoms N, O or S in its carbon skeleton. Examples of alkenyl groups are vinyl, allyl, but-1-enyl and but-2-enyl groups. Preferably an alkenyl group is straight-chained or branched and does not include any heteroatoms in its carbon skeleton. Preferably an alkenyl group is a C₂-C₁₂ alkenyl group, which is defined as an alkenyl group containing from 2 to 12 carbon atoms. More preferably an alkenyl group is a C₂-C₆ alkenyl group, which is defined as an alkenyl group containing from 2 to 6 carbon atoms. An ‘alkenylene’ group is similarly defined as a divalent alkenyl group.

An ‘alkynyl’ group is defined as a monovalent hydrocarbon, which comprises at least one carbon-carbon triple bond, which may be straight-chained or branched, or be or include cyclic groups. An alkynyl group may optionally include one or more heteroatoms N, O or S in its carbon skeleton. Examples of alkynyl groups are ethynyl, propargyl, but-1-ynyl and but-2-ynyl groups. Preferably an alkynyl group is straight-chained or branched and does not include any heteroatoms in its carbon skeleton. Preferably an alkynyl group is a C₂-C₁₂ alkynyl group, which is defined as an alkynyl group containing from 2 to 12 carbon atoms. More preferably an alkynyl group is a C₂-C₆ alkynyl group, which is defined as an alkynyl group containing from 2 to 6 carbon atoms. An ‘alkynylene’ group is similarly defined as a divalent alkynyl group.

An ‘aryl’ group is defined as a monovalent aromatic hydrocarbon. An aryl group may optionally include one or more heteroatoms N, O or S in its carbon skeleton. Examples of aryl groups are phenyl, naphthyl, anthracenyl and phenanthrenyl groups. Preferably an aryl group does not include any heteroatoms in its carbon skeleton. Preferably an aryl group is a C₄-C₁₄ aryl group, which is defined as an aryl group containing from 4 to 14 carbon atoms. More preferably an aryl group is a C₆-C₁₀ aryl group, which is defined as an aryl group containing from 6 to 10 carbon atoms. An ‘arylene’ group is similarly defined as a divalent aryl group.

For the purposes of the present invention, where a combination of groups is referred to as one moiety, for example arylalkyl, the last mentioned group contains the atom by which the moiety is attached to the rest of the molecule. A typical example of an arylalkyl group is benzyl.

For the purposes of this invention, an optionally substituted alkyl, aryl or arylalkyl group may be substituted with one or more of —F, —Cl, —Br, —I, —CF₃, —CCl₃, —CBr₃, —CI₃, —OH, —SH, —NH₂, —CN, —NO₂, —COOH, —R^{α}—O—R^{β}, —R^{α}—S—R^{β}, —R^{α}—SO—R^{β}, —R^{α}—SO₂—R^{β}, —R^{α}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}—CO—R^{β}, —R^{α}—CO—OR^{β}, —R^{α}O—CO—R^{β}, —R^{α}—CO—N(R^{β})₂, —R^{α}—NR^{β}—CO—R^{β}, —R^{α}O—CO—OR^{β}, —R^{α}O—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{a}—, or a π-bonded substituent such as ═O, ═S or ═NR^{β}. In this context, —R^{α}— is independently a chemical bond, or a C₁-C₁₀ alkylene, C₂-C₁₀ alkenylene or C₂-C₁₀ alkynylene group. —R^{β} is independently hydrogen, unsubstituted C₁-C₆ alkyl or unsubstituted C₆-C₁₀ aryl. Optional substituent(s) are preferably taken into account when calculating the total number of carbon atoms in the parent group substituted with the optional substituent(s). Preferably an optionally substituted alkyl, aryl or arylalkyl group is not substituted with a bridging substituent. Preferably an optionally substituted alkyl, aryl or arylalkyl group is not substituted with a π-bonded substituent. Preferably a substituted group comprises 1, 2 or 3 substituents, more preferably 1 or 2 substituents, and even more preferably 1 substituent.

As outlined above, the present invention provides novel methods for the preparation of sunitinib malate form I which is anhydrous, crystalline, non-hygroscopic, polymorphically stable, and wherein said methods have beneficial features that avoid the problems associated with prior art methods.

Accordingly, there is provided in a first detailed aspect a method for the preparation of sunitinib malate form I, comprising the steps of:

(i) mixing sunitinib with one or more solvents;

(ii) adding malic acid to the mixture from step (i); and

(iii) isolating a resultant solid from the mixture formed in step (ii).

In one embodiment, the sunitinib in step (i) is slurried in one or more solvents preferably selected from the group comprising acetone, methanol and ethyl acetate. Of course, it will be understood that there are a number of possible solvents that could be employed at this stage, for example, esters, ketones and hydroxylic solvents, the essential feature being that they result in the formation of anhydrous sunitinib malate crystalline form I. However, slurries can be prepared at chilled conditions, for example, at about 0-5° C. or 0-35° C., most preferably about 20-35° C. Slurries can also be prepared at elevated temperatures, above 35° C. One way is by using less volume of the slurrying solvent. Stirring and/or refluxing time may vary from 5 minutes to several hours.

Alternatively, the sunitinib is dissolved in one or more solvents preferably selected from the group comprising acetone, methanol and ethyl acetate. However, any of a number of solvents or solvent systems comprising miscible solvents and capable of dissolving sunitinib may be utilized. The skilled person will realize that there are number of means to encourage dissolution in a solvent, for example, by healing, agitating or sonication of the solvent mixture. The inventors have found that in particularly preferred embodiments heating sunitinib and the desired solvent to reflux temperatures is a particularly advantageous manner to effect dissolution of the sunitinib. In further preferred embodiments, the refluxing time may be varied from about 5 minutes to several hours or, in particularly preferred embodiments, is allowed to proceed until the slurry turns into a clear solution, indicating that the sunitinib is dissolved.

A further preferred embodiment provides that the malic acid in step (ii) is L- or D-malic acid, most preferably L-malic acid.

In yet another embodiment, the malic acid is dissolved in one or more solvents, preferably organic solvent(s), preferably methanol or alternatively water or acetone, before adding to the mixture formed in step (i). Of course, the skilled person will appreciate that any solvent or mixture of solvents that are capable of dissolving malic acid may be employed in the working of the invention.

In a further embodiment the malic acid is added to the sunitinib in step (ii) whilst stirring. The stirring can be carried out for about 5 minutes to several hours. In most embodiments, the stirring occurs until a slurry is observed, indicating that the desired sunitinib malate form I is present. The inventors have found that stirring for about 30 minutes is particularly advantageous.

Preferably the resultant slurry is further stirred and/or refluxed. In further preferred embodiments, wherein the mixture from steps (i) or (ii) has been heated, the solution or slurry is allowed to cool to about 15-35° C. The inventors have found that this temperature represents room temperature. Thus, there is no real need to reduce the temperature further to crystallize the sunitinib malate form I; however, if one wishes to do so, the solution or slurry may be further cooled or chilled to the desired temperature prior to isolation of the solid sunitinib malate form I. Preferably the slurry is stirred for a defined period of time.

In a particularly preferred embodiment, the solid sunitinib malate form I obtained in step (iii) is isolated by means of filtration and in some embodiments may be washed with the same solvent(s) as used in step (i). Washing in the same solvent(s) means that multiple solvents are not used, thus adding to the simplicity of the method and providing one of the advantages of the claimed invention.

The sunitinib malate form I may further be dried until a constant weight is achieved under conditions that do not degrade the solid sunitinib malate form I isolated or induce conversion to another polymorphic form. The inventors have found that drying on a rotavapour at reduced pressures facilitates drying at a temperature of about 30-50° C. Preferably the drying occurs at about 40° C. Certain embodiments wherein the rotavapour is used at near vacuum conditions have also been found to be particularly advantageous. Of course, it is within the skill set of the skilled person to determine the ideal conditions of isolating the compound during methods of the invention.

In a second detailed aspect according to the invention there is provided a method for the preparation of sunitinib malate form I, comprising the steps of:
(i) dissolving sunitinib in a solvent system wherein said solvent system comprises one or more solvents chosen from the group comprising acetone, methanol and ethyl acetate;
(ii) adding malic acid to the solution from step (i);
(iii) stirring the solution for a defined period of time; and
(iv) isolating the resultant solid sunitinib malate form I from the mixture formed in step (iii).

The skilled person will realize that there are number of means by which a solid may be encouraged to dissolve in a solvent or solvent system, for example, by heating, agitating or sonication of the solvent(s). The inventors have found that in particularly preferred embodiments sunitinib, dissolved in the solvent system of step (i) under reflux conditions or after being heated, is a particularly advantageous manner to effect dissolution of the sunitinib. In further preferred embodiments, the heating or refluxing time may be varied from about 5 minutes to several hours or, in particularly preferred embodiments, is allowed to proceed until the slurry turns to a clear solution, indicating that the sunitinib is dissolved.

A further preferred embodiment provides that the malic acid from step (ii) is L- or D-malic acid, most preferably L-malic acid.

In yet another embodiment, the malic acid is dissolved in one or more solvents, preferably organic solvent(s), most preferably methanol or alternatively water or acetone, before adding to the mixture formed in step (i). Of course, the skilled person will appreciate that any solvent or solvents that are capable of dissolving malic acid may be employed in the working of the invention.

In particularly preferred embodiments wherein the solution has been heated to effect dissolution of the sunitinib, the mixture from step (iii) is allowed to cool to ambient temperature, preferably about 15-35° C.

In a particularly preferred embodiment, the solid sunitinib malate form I from step (iv) is isolated by means of filtration and in alternative embodiments may be washed with the same solvent(s) as used in step (i). The sunitinib malate form I may further be dried until a constant weight is achieved under conditions that do not degrade the isolated solid sunitinib malate form I. Preferably the drying occurs at about 40° C. under reduced pressure, most preferably in a vacuum or partial vacuum.

In a third detailed aspect according to the invention there is provided an alternative method for the preparation of sunitinib malate form I, comprising the steps of:
(i) slurrying sunitinib in a solvent system wherein said solvent system comprises one or more solvents chosen from the group comprising acetone, methanol and ethyl acetate;
(ii) adding malic acid to the slurry from step (i);
(iii) stirring the slurry for a defined period of time; and
(iv) isolating the resultant solid sunitinib malate form I.

It will be understood that there are a number of possible solvents that could be employed during step (i), for example, esters, ketones and hydroxylic solvents, the essential feature being that they result in the formation of anhydrous crystalline form I. Slurries can be prepared at chilled conditions, for example, at about 0-5° C. or 0-35° C., most preferably about 20-35° C. Slurries can also be prepared at elevated temperatures, above 35° C. One way is by using less volume of the slurrying solvent Stirring and/or refluxing time may vary from 5 minutes to several hours.

A particularly preferred embodiment provides a method wherein one or more of the individual steps can be performed individually at about 10-35° C.

A further preferred embodiment provides that the malic acid from step (ii) is L- or D-malic acid, most preferably L-malic acid.

In a particularly preferred embodiment, the malic acid is added in step (ii) at ambient temperature, preferably at about 15-35° C.

In yet another embodiment, the malic acid is dissolved in one or more organic solvents, preferably methanol, alternatively water or acetone, before adding to the mixture formed in step (i). Of course, the skilled person will appreciate that any solvent or solvents that are capable of dissolving malic acid may be employed in the working of the invention.

In a particularly preferred embodiment, the solid sunitinib malate form I from step (iv) is isolated by means of filtration and in alternative embodiments may be washed with the same solvent(s) as used in step (i). The sunitinib malate form I may further be dried until a constant weight is achieved under conditions that do not degrade the solid sunitinib malate form I isolated. Preferably the drying occurs at about 30-50° C., most preferably at about 40° C. under reduced pressure, most preferably in a vacuum or partial vacuum.

The methods according to the invention provide sunitinib malate form I of great purity without the need for additional purification steps or techniques. Accordingly there is provided sunitinib malate form I when prepared according to any of the aspects or embodiments according to the invention. Preferably the sunitinib malate form I has a chemical and/or polymorphic purity of greater than 95%, preferably greater than 99%, more preferably greater than 99.5%, most preferably greater than 99.7%. Preferably the chemical purity is as measured by HPLC. Preferably the polymorphic purity is as measured by XRPD or DSC, preferably XRPD.

A fourth detailed aspect according to the invention provides a pharmaceutical composition comprising sunitinib malate form I prepared according to any of the aspects and embodiments according to the invention and disclosed herein. Preferably said pharmaceutical composition is provided for use in the treatment of cancer, in particular the treatment of cancer and tumors, and most preferably the treatment of unresectable and/or metastatic malignant gastrointestinal stromal tumor (GIST) or advanced and/or metastatic renal cell carcinoma (MRCC).

The pharmaceutical composition according to the present invention can be a solution or suspension, but is preferably a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a soft or hard shell, generally made of a gelatin material. Within said shell is generally comprised the active pharmaceutical ingredient formulated with or without pharmaceutically acceptable excipients into one of a number of compositions such as a powder, pellets, granules, mini-tablets and tablets in accordance with the invention.

The pharmaceutical composition according to the present invention typically comprises one or more conventional pharmaceutically acceptable excipient(s) selected from the group comprising a filler, a binder, a disintegrant, a lubricant, and optionally further comprises at least one excipient selected from colouring agents, adsorbents, surfactants, film-formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

Preferably the pharmaceutical compositions according to the fourth detailed aspect of the invention are for use in treating disorders related to abnormal protein kinase (PK) activity. Such diseases include, but are not limited to, diabetes, hepatic cirrhosis, cardiovascular disease such as atherosclerosis, angiogenesis, immunological disease such as autoimmune disease, malignant gastrointestinal stromal tumor (GIST) and metastatic renal cell carcinoma (MRCC).

The details of the invention, its objects and advantages are illustrated below in greater detail by non-limiting examples.

### EXAMPLES

Preparation of the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) in crystalline form I.

### Example 1

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was slurried in ethyl acetate (20 vol) at room temperature. L-Malic acid (1 eq) was added at a rate of 0.05 eq per minute under stirring until the formation of a slurry was observed. The slurry was stirred at room temperature (20-35° C.) for 30 minutes. The slurry was then filtered using a Buchner funnel under vacuum and the filtered solid washed with ethyl acetate (3 vol). The solid was then dried on a rotavapour at 40° C. under reduced pressure to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=83.30%.

HPLC purity=99.20%.

### Example 2

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was dissolved in ethyl acetate (60 vol) at reflux temperature. L-Malic acid (1 eq) was slowly added at a rate of 0.05 eq per minute whilst the solution was stirred. The formation of a slurry was observed. The slurry was refluxed for about 30 minutes and then gradually cooled to room temperature (20-35° C.). The slurry was stirred at this temperature for about 30 minutes. The slurry was then filtered with a Buchner funnel under vacuum and the resultant filtered solid dried on a rotavapour at 40° C. under reduced pressure until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=89.30%.

HPLC purity=99.41%.

### Example 3

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was slurried in ethyl acetate (15 vol) at room temperature. L-Malic acid (1 eq) dissolved in methanol (4 vol) was slowly added at a rate of 0.05 eq per minute to the slurry under stirring. The formation of a slurry was observed. The slurry was stirred at room temperature for 30 minutes and then filtered using a Buchner funnel under vacuum and the filtered solid washed with ethyl acetate (3 vol). The solid was then dried on a rotavapour at 40° C. until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=85.14%.

HPLC purity=99.12%.

### Example 4

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was dissolved in acetone (30 vol) at reflux temperature. L-Malic acid (1 eq) was slowly added at a rate of 0.05 eq per minute to the solution whilst stirring. The formation of a slurry was observed. The slurry was refluxed for about 15 minutes and then gradually cooled to room temperature (about 20-35° C.). The slurry was stirred at room temperature for about 15-30 minutes and then filtered using a Buchner funnel under vacuum. The filtered solid obtained was washed with acetone and then dried on a rotavapour at 40° C. under reduced pressure until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=90.90%.

HPLC purity=99.07%.

### Example 5

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was slurried in acetone (15 vol) at room temperature (25-30° C.). L-Malic acid (1 eq) dissolved in methanol (4 vol) was slowly added at a rate of 0.05 eq per minute to the mixture whilst stirring. The formation of a slurry was observed. The slurry was stirred at room temperature for about 30 minutes and then filtered using a Buchner funnel under vacuum and washed with acetone (3 vol). The filtered solid was dried on a rotavapour at 40° C. under reduced pressure until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=90.00%.

HPLC purity=99.37%.

### Example 6

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was dissolved in methanol (20 vol) at reflux temperature. L-Malic acid (1 eq) was slowly added at a rate of 0.05 eq per minute to the solution whilst stirring. The formation of a slurry was observed. The slurry was refluxed for about 15 minutes and then gradually cooled to room temperature (about 20-35° C.). The slurry was stirred at room temperature for about 15-30 minutes and then filtered using a Buchner funnel under vacuum and washed with methanol (3 vol). The filtered solid was dried on a rotavapour at 40° C. under reduced pressure until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=75.75%.

HPLC purity=99.23%.

### Example 7

N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) (1 eq) was slurried in methanol (10 vol) at room temperature (25-30° C.). L-Malic acid (1 eq) dissolved in methanol (4 vol) was slowly added at a rate of 0.05 eq per minute to the solution whilst stirring. A clear solution was observed. The stirring was continued for about 30 minutes and the formation of a slurry was observed. The slurry was stirred at room temperature for about 30 minutes and then filtered using a Buchner funnel under vacuum and washed with methanol (3 vol). The filtered solid was dried on a rotavapour at 40° C. under reduced pressure until a constant weight was achieved to obtain the L-malic acid salt of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (sunitinib) anhydrous crystal form I as a yellow solid.

Molar yield=75.75%.

HPLC purity=99.56%.

The anhydrous crystalline sunitinib malate form I obtained by following any of the examples 1-7 hereinbefore described exhibited the following analytical characteristics:

IR (KBr) cm⁻¹: 3326 (broad, N—H), 3231 (broad, O—H), 3063, 2927, 1671 (C═O), 1654, 1636, 1577, 1475, etc.

¹H-NMR (DMSO-d₆) ppm: 1.12 (t, J=7.14 Hz, 6H, 2x —CH₂CH₃), 2.36 (m, 2H, —CH₂—COOH), 2.44 (s, 3H, —CH₃), 2.46 (s, 3H, —CH₃), 2.55 (m, 1H, —CHOH—COOH), 2.92 (m, 6H, 3x —CH₂—), 4.02 (m, 2H, —CH₂—), 6.86 (m, 1H, vinyl proton), 6.94 (t, J=10.22 Hz, 1H, aromatic ortho position), 7.64 (br s, 1H, —CONH—, D₂O exchangeable), 7.73 (s, 1H, aromatic ortho position), 7.78 (d, J=9.42 Hz, 1H, aromatic meta position), 10.92 (s, 1H, —CONH—, D₂O exchangeable), 13.73 (s, 1H, pyrrole NH, D₂O exchangeable).

¹³C-NMR (DMSO-d₆) ppm: 9.69 (2C, 2x —CH₂—CH₃, DEPT), 10.68 & 13.46 (2C, 2x pyrrole —CH₃, DEPT), 35.01 (1C, —NH—CH₂—, DEPT), 40.89 (1C, —CHOH—), 46.81 (2C, 2x —CH₂—CH₃, DEPT), 50.57 (1C, Et₂N—CH₂—, DEPT), 66.40 (1C, —CH₂—COOH, DEPT), 106.06 (1C, d, J_{CF}=25.7 Hz, Ar—C meta position, DEPT), 110.08 (1C, d, J_{CF}=8.1 Hz, Ar—C ortho position, DEPT), 112.60 (1C, d, J_{CF}=24.9 Hz, Ar—C ortho position, DEPT), 115.04 (1C, bridgehead C adjacent to >NH), 119.90 & 125.90 & 134.50 & 136.96 (4C, pyrrole), 124.91 (1C, ═CH—, DEPT), 127.10 (1C, d, J_{CF}=9.7 Hz, bridgehead C adjacent to >C═), 130.30 (1C, >C═CH—), 158.36 (1C, d, J_{CF}=234.4 Hz, —CF═), 165.30 & 169.52 (2C, 2x —NH—CO—), 172.21 & 176.06 (2C, 2x —COOH).

Mass (m/z): (M+1) 399 (100%), [(M+2)+1] 401 (14%).

XRPD: 12.94, 19.15, 23.94, and 25.20.

DSC: 195° C.

The XRPD data given above and the spectrum of FIG. 1 were recorded on a Bruker D8 Advance Instrument, using Cu α-radiation as the X-ray source, with a 2θ range of from 3 to 50°, a step-size of 0.5° and a time/step of 1 sec.

1.-80. (canceled)

81. A method for the preparation of sunitinib malate form I, comprising the steps of:
mixing sunitinib with one or more solvents;
adding malic acid to the mixture from step (i); and
isolating a resultant solid from the mixture formed in step (ii).

82. A method according to claim 81, wherein:
the sunitinib in step (i) is dissolved or slurried in one or more solvents selected from the group comprising acetone, methanol and ethyl acetate; and/or
the sunitinib in step (i) is slurried in one or more solvents selected from the group comprising acetone, methanol and ethyl acetate; and/or
the sunitinib in step (i) is slurried at about 15-35° C. in one or more solvents selected from the group comprising acetone, methanol and ethyl acetate; and/or
the sunitinib in step (i) is dissolved in one or more solvents selected from the group comprising acetone, methanol and ethyl acetate; and/or
the sunitinib in step (i) is dissolved at about reflux temperature in one or more solvents selected from the group comprising acetone, methanol and ethyl acetate; and/or
the malic acid in step (ii) is L- or D-malic acid; and/or
the malic acid in step (ii) is L-malic acid; and/or
the malic acid is dissolved in one or more solvents before addition to the mixture from step (i); and/or
the malic acid is dissolved in methanol before addition to the mixture from step (i); and/or
the malic acid is added to the sunitinib in step (ii) whilst stirring; and/or
the malic acid is added to the sunitinib in step (ii) at a rate of about 0.05 equivalents of malic acid per minute; and/or
the resultant solid isolated in step (iii) is sunitinib malate form I.

83. A method according to claim 81, wherein after the addition of the malic acid or the sunitinib in step (ii):
a further step (ii-a) of heating the mixture is performed before the isolation of step (iii) occurs; and/or
a further step (ii-a) of heating the mixture to about the reflux temperature of the solvent or solvent mixture is performed before the isolation of step (iii) occurs; and/or
a further step (ii-a) of heating the mixture for about 15-30 minutes is performed before the isolation of step (iii) occurs.

84. A method according to claim 81, wherein after the addition of the malic acid or the sunitinib in step (ii) and, if present, after the heating of the mixture in step (ii-a):
a further step (ii-b) of allowing the mixture to stand for a period of at least 5 minutes is performed before the isolation of step (iii) occurs; and/or
a further step (ii-b) of allowing the mixture to stand for a period of about 15-30 minutes is performed before the isolation of step (iii) occurs; and/or
a further step (ii-b) of allowing the mixture to stand at a temperature of about 20-35° C. for a period of at least 5 minutes is performed before the isolation of step (iii) occurs; and/or
a further step (ii-b) of allowing the mixture to stand with stirring for a period of at least 5 minutes is performed before the isolation of step (iii) occurs.

85. A method according to claim 81, wherein after the addition of the malic acid or the sunitinib in step (ii):
a further step of stirring the mixture is performed until a slurry is formed; and/or
a further step of stirring the mixture is performed until a slurry is formed, wherein the slurry is heated or refluxed; and/or
a further step of stirring the mixture is performed until a slurry is formed, wherein the slurry is heated or refluxed and then allowed to cool to about 0-35° C.; and/or
a further step of stirring the mixture is performed until a slurry is formed, wherein the slurry is stirred for a defined period of time.

86. A method according to claim 81, wherein:
the solid sunitinib from step (iii) is isolated by means of filtration; and/or
the solid isolated in step (iii) is washed with the same solvent(s) as used in step (i); and/or
the solid isolated in step (iii) is dried until a constant weight is achieved, preferably under conditions that do not degrade the solid obtained; and/or
the solid isolated in step (iii) is dried at about 40° C. under reduced pressure until a constant weight is achieved.

87. A method for the preparation of sunitinib malate form I, comprising the steps of:
mixing malic acid with one or more solvents;
adding sunitinib to the mixture from step (i); and
isolating a resultant solid from the mixture formed in step (ii).

88. A method according to claim 87, wherein:
the sunitinib is dissolved or slurried in one or more solvents before addition to the mixture from step (i); and/or
the sunitinib is slurried in one or more solvents selected from the group comprising acetone, methanol and ethyl acetate; and/or
the sunitinib is slurried at about 15-35° C. in one or more solvents selected from the group comprising acetone, methanol and ethyl acetate; and/or
the sunitinib is dissolved in one or more solvents selected from the group comprising acetone, methanol and ethyl acetate; and/or
the sunitinib is dissolved at about reflux temperature in one or more solvents selected from the group comprising acetone, methanol and ethyl acetate; and/or
the malic acid in step (i) is L- or D-malic acid; and/or
the malic acid in step (i) is L-malic acid; and/or
the malic acid is dissolved in methanol; and/or
the malic acid is dissolved in methanol at about reflux temperature; and/or
the sunitinib is added to the malic acid in step (ii) whilst stirring; and/or
the sunitinib is added to the malic acid in step (ii) at a rate of about 0.05 equivalents of sunitinib per minute; and/or
the resultant solid isolated in step (iii) is sunitinib malate form I.

89. A method according to claim 87, wherein after the addition of the malic acid or the sunitinib in step (ii):
a further step (ii-a) of heating the mixture is performed before the isolation of step (iii) occurs; and/or
a further step (ii-a) of heating the mixture to about the reflux temperature of the solvent or solvent mixture is performed before the isolation of step (iii) occurs; and/or
a further step (ii-a) of heating the mixture for about 15-30 minutes is performed before the isolation of step (iii) occurs.

90. A method according to claim 87, wherein after the addition of the malic acid or the sunitinib in step (ii) and, if present, after the heating of the mixture in step (ii-a):
a further step (ii-b) of allowing the mixture to stand for a period of at least 5 minutes is performed before the isolation of step (iii) occurs; and/or
a further step (ii-b) of allowing the mixture to stand for a period of about 15-30 minutes is performed before the isolation of step (iii) occurs; and/or
a further step (ii-b) of allowing the mixture to stand at a temperature of about 20-35° C. for a period of at least 5 minutes is performed before the isolation of step (iii) occurs; and/or
a further step (ii-b) of allowing the mixture to stand with stirring for a period of at least 5 minutes is performed before the isolation of step (iii) occurs.

91. A method according to claim 87, wherein after the addition of the malic acid or the sunitinib in step (ii):
a further step of stirring the mixture is performed until a slurry is formed; and/or
a further step of stirring the mixture is performed until a slurry is formed, wherein the slurry is heated or refluxed; and/or
a further step of stirring the mixture is performed until a slurry is formed, wherein the slurry is heated or refluxed and then allowed to cool to about 0-35° C.; and/or
a further step of stirring the mixture is performed until a slurry is formed, wherein the slurry is stirred for a defined period of time.

92. A method according to claim 87, wherein:
the solid sunitinib from step (iii) is isolated by means of filtration; and/or
the solid isolated in step (iii) is washed with the same solvent(s) as used in step (i); and/or
the solid isolated in step (iii) is dried until a constant weight is achieved, preferably under conditions that do not degrade the solid obtained; and/or
the solid isolated in step (iii) is dried at about 40° C. under reduced pressure until a constant weight is achieved.

93. A method for the preparation of sunitinib malate form I, comprising the steps of:
dissolving sunitinib in a solvent system at elevated temperature wherein said solvent system comprises one or more solvents chosen from the group comprising acetone, methanol and ethyl acetate;
adding malic acid to the solution from step (i);
stirring the solution for a defined period of time; and
isolating the resultant solid sunitinib malate form I from the mixture formed in step (iii).

94. A method according to claim 93, wherein:
the sunitinib is dissolved in the solvent system from step (i) under reflux conditions; and/or
the malic acid in step (ii) is L- or D-malic acid; and/or
the malic acid in step (ii) is L-malic acid; and/or
the malic acid is dissolved in one or more solvents before addition to the solution from step (i); and/or
the malic acid is dissolved in methanol before addition to the solution from step (i); and/or
the malic acid is added to the sunitinib in step (ii) whilst stirring; and/or
the resultant solid sunitinib malate form I is isolated from the mixture formed in step (iii) by filtration; and/or
the solid isolated in step (iv) is washed with the same solvent(s) as used in step (i); and/or
the solid isolated in step (iv) is dried until a constant weight is achieved, preferably under conditions that do not degrade the solid obtained; and/or
the solid isolated in step (iv) is dried at about 40° C. under reduced pressure until a constant weight is achieved.

95. A method for the preparation of sunitinib malate form I, comprising the steps of:
slurrying sunitinib in a solvent system wherein said solvent system comprises one or more solvents chosen from the group comprising acetone, methanol and ethyl acetate;
adding malic acid to the slurry from step (i);
stirring the slurry for a defined period of time; and
isolating the resultant solid sunitinib malate form I.

96. A method according to claim 95, wherein:
one or more of the individual steps is performed individually at about 15-35° C.; and/or
the malic acid from step (ii) is L- or D-malic acid; and/or
the malic acid from step (ii) is L-malic acid; and/or
the malic acid is dissolved in one or more solvents before addition to the slurry from step (i); and/or
the malic acid is dissolved in methanol before addition to the slurry from step (i); and/or
the malic acid is added to the sunitinib in step (ii) whilst stirring; and/or
the resultant solid sunitinib malate form I is isolated in step (iv) by filtration; and/or
the solid isolated in step (iv) is washed with the same solvent(s) as used in step (i); and/or
the solid isolated in step (iv) is dried until a constant weight is achieved, preferably under conditions that do not degrade the isolated solid sunitinib malate form I; and/or
the solid isolated in step (iv) is dried at about 40° C. under reduced pressure until a constant weight is achieved.

97. Sunitinib malate form I when prepared by a method according to claim 81.

98. Sunitinib malate form I when prepared by a method according to claim 87.

99. Sunitinib malate form I when prepared by a method according to claim 93.

100. Sunitinib malate form I when prepared by a method according to claim 95.

101. Sunitinib malate form I according to claim 97, having a chemical and/or polymorphic purity of:
greater than 95%;
greater than 99%;
greater than 99.5%; or
greater than 99.7%.

102. Sunitinib malate form I according to claim 98, having a chemical and/or polymorphic purity of:
greater than 95%;
greater than 99%;
greater than 99.5%; or
greater than 99.7%.

103. Sunitinib malate form I according to claim 99, having a chemical and/or polymorphic purity of:
greater than 95%;
greater than 99%;
greater than 99.5%; or
greater than 99.7%.

104. Sunitinib malate form I according to claim 100, having a chemical and/or polymorphic purity of:
greater than 95%;
greater than 99%;
greater than 99.5%; or
greater than 99.7%.

105. A pharmaceutical composition comprising sunitinib malate form I according to claim 97.

106. A pharmaceutical composition comprising sunitinib malate form I according to claim 98.

107. A pharmaceutical composition comprising sunitinib malate form I according to claim 99.

108. A pharmaceutical composition comprising sunitinib malate form I according to claim 100.

109. A pharmaceutical composition according to claim 105, for the treatment of:
a tumor; and/or
cancer; and/or
unresectable and/or metastatic malignant gastrointestinal stromal tumor (GIST) or advanced and/or metastatic renal cell carcinoma (MRCC).

110. A pharmaceutical composition according to claim 106, for the treatment of:
a tumor; and/or
cancer; and/or
unresectable and/or metastatic malignant gastrointestinal stromal tumor (GIST) or advanced and/or metastatic renal cell carcinoma (MRCC).

111. A pharmaceutical composition according to claim 107, for the treatment of:
a tumor; and/or
cancer; and/or
unresectable and/or metastatic malignant gastrointestinal stromal tumor (GIST) or advanced and/or metastatic renal cell carcinoma (MRCC).

112. A pharmaceutical composition according to claim 108, for the treatment of:
a tumor; and/or
cancer; and/or
unresectable and/or metastatic malignant gastrointestinal stromal tumor (GIST) or advanced and/or metastatic renal cell carcinoma (MRCC).

113. A method of treating a tumor, or cancer, or unresectable and/or metastatic malignant gastrointestinal stromal tumor (GIST) or advanced and/or metastatic renal cell carcinoma (MRCC), comprising administering to a patient in need thereof a therapeutically effective amount of sunitinib malate form I according to claim 97.

114. A method of treating a tumor, or cancer, or unresectable and/or metastatic malignant gastrointestinal stromal tumor (GIST) or advanced and/or metastatic renal cell carcinoma (MRCC), comprising administering to a patient in need thereof a therapeutically effective amount of sunitinib malate form I according to claim 98.

115. A method of treating a tumor, or cancer, or unresectable and/or metastatic malignant gastrointestinal stromal tumor (GIST) or advanced and/or metastatic renal cell carcinoma (MRCC), comprising administering to a patient in need thereof a therapeutically effective amount of sunitinib malate form I according to claim 99.

116. A method of treating a tumor, or cancer, or unresectable and/or metastatic malignant gastrointestinal stromal tumor (GIST) or advanced and/or metastatic renal cell carcinoma (MRCC), comprising administering to a patient in need thereof a therapeutically effective amount of sunitinib malate form I according to claim 100.
